(19) United States
(12) Patent Application Publication
(10) Pub. No.: US 2006/0084704 A1
(43) Pub. Date: Apr. 20, 2006

(54) Methods and compositions for enhancing degradation of nuclear receptor transcription factors and uses thereof

(76) Inventors: Charles Shih, Solana Beach, CA (US); Ching-Yuan Su, San Diego, CA (US)

Correspondence Address: DAVID R PRESTON & ASSOCIATES APC, 12625 HIGH BLUFF DRIVE, SUITE 205, SAN DIEGO, CA, 92130, US

(21) Appl. No.: 11/218,889

(22) Filed: Sep. 2, 2005

Related U.S. Application Data
(63) Continuation-in-part of application No. 11/045,181, filed on Jan. 27, 2005.
(60) Provisional application No. 60/539,753, filed on Jan. 28, 2004.
(60) Provisional application No. 60/606,678, filed on Sep. 2, 2004.

(51) Int. Cl. A61K 9/127; A61K 31/12

(52) U.S. Cl. 514/688; 424/450

(57) ABSTRACT

The present invention includes methods, compositions, cosmetics and pharmaceutical compositions for enhancing the degradation of a nuclear receptor (NR) or a STAT transcription factor protein. The methods, compositions, cosmetics and pharmaceuticals may be used to prevent or treat disorders or medical conditions that are at least in part affected by a nuclear receptor activation pathway or STAT activation pathway.

This application is a continuation-in-part of U.S. patent application Ser. No. 11/045,181 filed on Jan. 27, 2005, which claims priority to U.S. provisional patent application Ser. No. 60/539,753 filed on Jan. 28, 2004. This application also claims benefit of priority to U.S. provisional patent application Ser. No. 60/606,678 filed on Sep. 2, 2004. Each application referred to is incorporated by reference in its entirety.

This invention was made with government support awarded by the National Institutes of Health, National Cancer Institute, grant numbers 1R43 CA96189-01 and 1R41 CA97647-01. The United States Government may have certain rights in this invention.

### FIELD OF THE INVENTION

The present invention relates generally to the field of medicine, and particularly to methods for degradation of transcription factors, especially steroid nuclear hormone receptors such as the androgen receptor (AR), progesterone receptor (PR), and estrogen receptors α and β (ER), and uses thereof.

### BACKGROUND OF THE INVENTION

Androgen exerts its function by entering a target cell and binding to a specific androgen receptor (AR), leading to the activation of androgen-regulating genes. The male circulating androgen hormone, testosterone, is converted within cells of peripheral tissues by 5-alpha reductase enzymes to the main intracellular androgen, dihydrotestosterone (DHT). There are available anti-androgen drugs that either inhibit the conversion of testosterone into DHT (for example, 5-alpha-reductase inhibitors) or that interfere with the binding between androgen and AR, but these anti-androgen drugs can cause side effects, such as impotence in some men receiving treatment.

The androgen-induced AR activation pathway is a multiple step process (see, for example, Lee and Chang (2003) J. Clin. Endocrinol. Metab., 88:4043-4054, which is incorporated by reference in its entirety herein) and is believed to not simply be limited to androgen and AR binding. The AR activation pathway involves AR protein expression and AR protein degradation, and in addition, involves androgen-AR complex (A-AR complex) formation, A-AR complex phosphorylation, A-AR complex translocation from cytoplasm into nucleus, and A-AR complex and AR coregulator complex (A-AR-ARA) formation (see, for example, Lin et al. (2002) EMBO J., 21:4037-4048, which is incorporated by reference in its entirety herein). Within the nucleus, the androgen-AR-ARA complex is believed to function as a transcription factor, binding to the androgen response element (ARE) on the promoter region of androgen-regulated gene(s) and recruiting other regulatory proteins involved in general transcription machinery that lead to the activation (expression or repression) of androgen-responsive target gene(s).

Two isoforms (the full-length AR-B and the N-terminus truncated AR-A) of the androgen receptor are expressed in immunologically detectable forms in many fetal and adult human tissues (Wilson and McPhaul (1996)). High AR levels are found in both male and female fetal reproductive tissues, and in varying levels in non-genital fetal tissues. High AR levels are also found in adult reproductive tissues (prostate, endometrium, ovary, uterus, fallopian tube, testis, seminal vesicle, myometrium, and ejaculatory duct), and lower levels in adult breast, colon, lung and adrenal gland tissue. The AR pathway is especially important in the development and proper function of the male reproductive organs as well as non-reproductive organs (including muscle, hair follicles, and the brain). It is involved in the pathology of several diseases or conditions, including prostate cancer and other cancers, male infertility, and Kennedy disease.

Prostate cancer is the most common malignancy in American men in terms of incidence and prevalence. It is the most frequently diagnosed neoplasm in the United States and the second leading cause of cancer-related death for American men (Boring et al., 1992). Prostate cancer strikes more than 180,000 men each year, about the same number as cases of breast cancer in women. It caused 31,900 deaths among American men in 1999, second only to lung cancer. The increase in incidence of prostate cancer each year correlates with the aging of American male population.

Prostate cancer cell growth is believed to rely upon androgen-induced activation of the androgen receptor (AR). Most prostate cancers are dependent on androgen when first diagnosed (Heinlein and Chang 2004), and thus can be treated with anti-androgens. One effective treatment for metastatic prostate cancer is androgen blockage therapy, which employs either surgical or chemical castration, combined with anti-androgen treatment, to suppress the biological action of androgens (Crawford et al., 1989). However, the median duration of cancer response to hormone depletion is only 18-36 months, and the cancer almost always relapses and becomes androgen non-responsive. In such cases, patients face less desirable therapies, such as chemotherapy. In some cases, alterations of anti-androgens delay the progression of recurrent prostate cancers (Dupont et al, 1993; Taplin et al., 1999), indicating that a prostate tumor or cancer which relapses on a specific anti-androgen therapy may respond to a different anti-androgen.

Kennedy Disease—also known as Kennedy Disease, spinal and bulbar muscular atrophy or spinobulbar muscular atrophy (“SBMA”), or Kennedy Syndrome (see, for example, Paul E. Barkhaus (2003), “Kennedy Disease”, electronic publication available on-line at http://www.emedicine.com/neuro/topic421.htm, accessed 15 Apr. 2004)—is a rare, X-linked recessive genetic neuromuscular disease that is estimated to affect 1 in 40,000 individuals worldwide. It is progressive, and currently incurable and non-treatable. Both the spinal and bulbar neurons are affected, causing muscle weakness and atrophy throughout the body, most noticeably in the extremities and in the face and throat. Kennedy Disease causes speech and swallowing difficulties, major muscle cramps, as well as other symptoms. It is an adult-onset disease with symptoms usually appearing between the ages of 30 and 50, although earlier onsets have been recorded. Only males with this inherited gene develop the full phenotype of the disease, whereas females heterozygous for the gene are generally asymptomatic carriers. In some cases, females who are heterozygous for Kennedy Disease show subclinical phenotypic expression. Life expectancy is generally not affected.

Kennedy disease is believed to be caused by an androgen receptor mutation consisting of an abnormally long polyglutamine expansion in the N-terminus region of the AR gene. Experimental transfection of cells with a mutated AR having expanded polyglutamine repeats (for example, with the plasmids p6RARQ49 or p6RARQ77) has been shown to be associated with a decreased transactivational function and, in some cases, intranuclear inclusions of misfolded AR proteins (Chamberlain et al. (1994) Nucleic Acid Res., 22:3181-3186, which is incorporated by reference in its entirety herein). This intranuclear accumulation of abnormal AR is cytotoxic, triggering neuronal cell death, consistent with the in vivo pathology of Kennedy disease.

### BRIEF SUMMARY OF THE INVENTION

The present invention addresses the need for new methods of treating or preventing disease or medical conditions that are, at least in part, affected by the activity of one or more transcription factors such as nuclear receptors or STAT transcription factor proteins. Thus, one aspect of the present invention includes a method of inhibiting or reducing the effect of a nuclear receptor activation pathway is disclosed including providing a cell including a nuclear receptor activation pathway and introducing a compound capable of inducing, enhancing or increasing degradation of the desired nuclear receptor. The nuclear receptor pathway may include one or more transcription factors, cofactors, coregulators, corepressors or signalling pathways. Each of which may be affected using the disclosed methods. Degradation of the nuclear receptor may include interfering with phosporylation of a transcription factor or cofactor, interfering with dimerization (homo- or heterodimerization) of a transcription factor or cofactor, interfering with binding between a trascription factor and a cofactor, interfering with nuclear transfer of a transcription factor or cofactor and the like.

In another aspect of the present invention a method of inhibiting or reducing the effect of a STAT activation pathway is disclosed including providing a cell including a STAT activation pathway and introducing a compound capable of inducing, enhancing or increasing the degradation of a STAT transcription factor protein.

In other aspects of the present invention pharmaceutical or cosmetic compositions are provided that are capable of modulating the effect of a nuclear receptor activation pathway or a STAT activation pathway by enhancing or increasing degradation of a transcription factor, a nuclear receptor or a STAT transcription factor protein by any one or more of a variety of mechanisms.

In another aspect of the present invention methods of treating a medical condition or a cause or symptom of a medical condition are disclosed including administering a cosmetic composition or pharmaceutical composition capable of enhancing or inducing degradation of a transcription factor such as a nuclear receptor or a STAT transcription factor protein to an individual suffering from or at risk of developing a nuclear receptor related or STAT related medical condition. Nonlimiting examples of conditions that may be treated with the disclosed pharmaceuticals or cosmetics include male infertility, Kennedy disease, prostate cancer, breast cancer, liver cancer, bladder cancer, benign prostate hyperplasia, acne, baldness, hirsutism, exposed wounds and unwanted preganancy.

### BRIEF DESCRIPTION OF THE DRAWINGS

FIG. 1 depicts a graphical representation of the results of an androgen receptor transactivation assay as described in Example 2 using CV-1, green monkey kidney cells (FIG. 1A) and LNCaP, human prostate cancer cells (FIG. 1B). Referring to FIG. 1A, Non-androgen expressing CV-1 cells were transfected with a wild type androgen receptor expression vector utilizing MMTV as a reporter gene and pRL as an internal transfection control. DHT was added at 1 nM and compound JC9 was provided at varying concentrations from 0 uM to 7.5 uM. Cell lysates were collected, enzyme activities assayed and the results were compared using percent (%) relative luciferase activity. As indicated by the graph in FIG. 1A, compound JC9 was capable of inhibiting DHT-induced androgen receptor transcriptional activation in a dose dependent manner. Referring to FIG. 1B, LNCaP cells, which express a mutant androgen receptor, were transfected with MMTV and prL. As in FIG. 1A, DHT was provided at 1 nM and compound JC9 was provided from 0 uM to 7.5 uM. Again, a dose-dependent inhibition of LNCaP androgen receptor transactivation was observed in cells incubated with JC9 compound.

FIG. 2 depicts a graphical representation of cell growth (proliferation) and androgen receptor expression levels in JC9-treated LNCaP cells. LNCaP cells were plated and incubate for two days. JC9 and hydroxyflutamide (HF) were added, separately, to the medium at a final concentration of 5 uM with and without DHT. Referring to FIG. 2A, the results demonstrate that while DHT promote LNCaP cell growth in culture, JC9 significantly inhibits cell growth regardless in the presence or absence of DHT. On the other hand, HF only moderately inhibits cell growth in either condition. FIG. 2B depicts normalized androgen receptor signals for the JC9 sample as a percentage of baseline (Day 0) value. Cell lysates collected from cell cultured with JC9 (FIG. 2A) and AR expression was detected by Western Blot. Data indicated that the inhibition of AR expression in LNCaP cells, induced by JC-9, is in correlation with cell growth inhibition.

FIG. 3 depicts a western blots analysis of LNCaP cell lysates that were cultured with JC15 for 20 hr. in the presence or absence of DHT. Data demonstrated that JC15 reduced AR, PR and PSA proteins expression regardless in the presence or absence of DHT, but did not affect expression of other proteins, such as GR, ER, PPAR, RXR, HSP and Actin.

FIG. 4 depicts a western blot analysis of T47D (a human breast cancer) cell lysates demonstrating the specificity of JC9's ability to degrade the androgen receptor. Data demonstrated that JC9 selectively reduced expression of androgen receptor (AR). The expression of other receptor proteins, Peroxisome proliferator-activated receptors gamma and beta (PPARγ, PPARβ), retinoid X receptor alpha (RXRα), estrogen receptor alpha and beta (ERα and Erβ), extracellular signal-related kinase (ERK), heat shock protein 70 (HSP70) and actin, was not affected.

FIG. 5 depicts a western blot analysis of LNCaP cell lysates upon exposure to compound JC9 and cyclohexamide, a protein synthesis inhibitor. The reduction of androgen receptor over time in the presence of a protein synthesis inhibitor indicates JC9 enhances degradation of AR protein.

FIG. 6 depicts fluorescence micrographs of monkey kidney COS-1 cells transfected with the plasmid GFPARQ49 (which contained a green fluorescent protein as a reporter and the mutant androgen receptor Q49) as described in detail in Example 5. Transfected cells were treated with vehicle only (control) or with the test compound JC9. Micrographs were taken under fluorescent imaging conditions for green fluorescent protein (GFP) and propidium iodide (PI). Control cells contained large amounts of fluorescent inclusions or aggregates. Cells that had been treated with JC9 contained substantially smaller amounts of fluorescent inclusions, suggesting that the expression of mutant Q49 androgen receptor was inhibited or degraded by JC9 treatment.

FIG. 7 depicts photographs demonstrating a representative visible improvement to skin condition resulting from topical application of the test compound JC15 (1 micromolar in a carrier base) to the forehead of an acne-affected male volunteer, as described in detail in Example 6. FIG. 7A depicts the volunteer's forehead prior to starting treatment with JC15. FIG. 7B depicts the same volunteer's forehead after one month of topical treatment with JC15

FIG. 8 depicts representative photographs of Fuzzy rats treated as described in detail in Example 7. Fuzzy rats were treated with topical creams containing vehicle only (left side animal) or JC9 (25 micromolar, right side animal) for the times indicated. The photographs show that bands of sebaceous glands were reduced within 4-5 weeks in the Fuzzy rats treated with JC9 (right side animal).

FIG. 9 depicts representative photographs (FIGS. 9A-B) and graphical representations of duct and lobe size (FIGS. 9C-E) of sebaceous glands in Fuzzy rat skin. Skin tissue samples (split skin) were prepared and examined by microscopy. FIG. 9A-B are photographs depicting the duct and lobe of the sebaceous gland upon treatment with a vehicle control (9A) or compound JC9 (9B). In FIG. 9C, size of glandular lobes were measured by tracing the edges of the glandular lobes from areas of well-preserved glandular lobules, and then quantified with Image J software, and expressed as pixel counts contained within the traced areas. The data obtained showed that the size of the sebaceous glandular lobe is approximately twice in male Fuzzy rats as in females. Topical treatment with the vehicle only (control cream) did not produce a significant change in glandular lobe size. Topical treatment of male rats with the test compounds JC15 and JC9 resulted in a reduction in the size of the sebaceous glandular lobe, with the decrease caused by JC15 approximately equivalent to the decrease caused by castration. FIGS. 9D and 9E depict representative data showing that JC9 applied to skin in a vehicle cream significantly reduced the size of lobe and ducts of sebaceous glands in male Fuzzy rats.

FIG. 10A depicts results from studies of an animal model of alopecia (hair loss or baldness), as described in detail in EXAMPLE 8. Six-week-old male C57BL/6J mice were shaved with an electric clipper, and then treated with a hair-removal cream. One group of mice, represented by the two left-most animals (marked “vehicle #1” and “vehicle #2”) were shaved and treated only with ethanol. A second group of mice, represented by the two right-most animals (marked “testosterone #1” and “testosterone #2”) were shaved and treated with a testosterone/ethanol solution in the morning and a control solution in the afternoon. The animals were photographed at the end of the 20-day treatment period. Mice treated with the ethanol vehicle alone (without testosterone) showed rapid re-growth of hair in the shaved areas after 20 days of topical treatment. Mice treated with testosterone showed little or no re-growth of hair in the shaved areas after 20 days of topical treatment. FIG. 10B depicts further results from studies of an animal model of alopecia (hair loss or baldness), as described in detail in EXAMPLE 8. Six-week-old male C57BL/6J mice were shaved with an electric clipper, and then treated with a hair-removal cream. One group of mice, (represented by the animals marked “testosterone #1” and “testosterone #2”) were shaved and treated topically with testosterone in the morning and control solution in the afternoon for twenty days. A second group of mice (represented by the animals marked “JC9/testosterone #1” and “JC9/testosterone #2”) were shaved and treated topically with testosterone in the morning and JC9 in the afternoon for twenty days. Mice that received topical morning applications of testosterone and afternoon applications of the control solution only showed little or no re-growth of hair in the shaved areas after 20 days of treatment. Mice that received topical morning applications of testosterone and afternoon applications of JC9 showed hair growth on day 8 and fully re-growth of hair in the shaved areas after 20 days of topical JC9 treatment. These results demonstrate that topical application of JC9, is able to overcome testosterone-induced hair growth suppression in an animal model.

FIG. 11 depicts a photograph of nude mice inoculated with 2 million LNCaP cells. The mice were given an intrapertioneal injection with either JC9 or a vehicle control three times per week for 7 weeks. After 7 weeks, the tumor was excised and weighed. The JC9 treated nude mouse demonstrated a 75% reduction in tumor size compared to tumor excised from the mouse treated with vehicle control, indicated JC9 compound is capable of inhibits tumor cell growth in vivo.

### DETAILED DESCRIPTION OF THE INVENTION

The present invention makes use of a cell-based, functional assay system that measures reporter gene activity in order to identify novel compounds that inhibit a nuclear receptor pathway (or the androgen-induced androgen receptor (AR) activation pathway). Preferably, these novel compounds exert their anti-hormone activity through an inhibition mechanism different from that of the conventional 5-alpha-reductase inhibitors or androgen-AR binding inhibitors. Active novel compounds have been identified, including, but not limited to, chemical derivatives and analogues of the natural compound, curcumin (diferuloylmethane), purified from the turmeric plant, Curcuma longa (see, for example, Ohtsu H. et al. (2002), J. Med. Chem., 45:5037-5024, which is incorporated by reference in its entirety herein). Non-limiting examples of these curcumin derivatives include the JC series of compounds, such as JC9 (5-hydroxy-1,7-bis(3,4-dimethoxyphenyl)-1,4,6-heptatrien-3-one) and JC15 (7-(4-hydroxy-3-methoxyphenyl)-4-[3-(4-hydroxy-3-methoxyphenyl)acryloyl]-5-oxo-hept-6-enoic acid ethyl ester), whose structures and preparations are described in Ohtsu et al. (2002), J. Med. Chem., 45:5037-5042 and Ohtsu et al. (2003) Bioorg. Med. Chem., 11:5083-5090, which are incorporated by reference in their entirety herein. Unlike the parent natural product, curcumin, these novel derivatives possess significant in vitro and in vivo anti-androgenic activity at pharmacologically achievable doses.

The present invention recognizes that specific degradation of a nuclear receptor is a novel and useful mechanism for modulating effects of a nuclear receptor-activated pathway. The present invention answers the need for, and provides, new therapeutic approaches for preventing and treating disease conditions that are, at least in part, affected by the activity of a nuclear receptor, such as, but not limited to, steroid hormone receptors including the androgen receptor and the progesterone receptor. The invention provides methods for treating or preventing such disease conditions in a subject. The invention also provides methods for screening for compounds useful in the treatment or prevention of such disease conditions and disorders. The invention further provides compositions useful in the treatment or prevention of such disease conditions.

As a non-limiting introduction to the breadth of the present invention, the present invention includes several useful aspects, including:
1. A method of enhancing or inducing degradation of a nuclear receptor or a STAT transcription factor protein.
2. A pharmaceutical or cosmetic composition capable of enhancing or inducing degradation of a nuclear receptor or STAT transcription factor protein and thereby preventing or treating a cause or symptom of disease conditions that are, at least in part, affected by the activity of the nuclear receptor or STAT transcription factor protein.
3. A method to prevent or treat causes or symptoms of medical or disease conditions that are, at least in part, affected by the activity of a nuclear receptor or STAT transcription factor protein, by enhancing or inducing degradation of the nuclear receptor or STAT transcription factor protein.

Further objectives and advantages of the present invention will become apparent as the description proceeds and when taken in conjunction with the accompanying drawings. To gain a full appreciation of the scope of the present invention, it will be further recognized that various aspects of the present invention can be combined to make desirable embodiments of the invention.

Throughout this application various publications are referenced. The disclosures of these publications are hereby incorporated by reference, in their entirety, in this application. Citations of these documents are not intended as an admission that any of them are pertinent prior art. All statements as to the date or representation as to the contents of these documents is based on the information available to the applicant and does not constitute any admission as to the correctness of the dates or contents of these documents.

Unless defined otherwise, all technical and scientific terms used herein have the same meaning as commonly understood by one of ordinary skill in the art to which this invention belongs. Generally, the nomenclature used herein and the manufacture or laboratory procedures described below are well known and commonly employed in the art. The technical terms used herein have their ordinary meaning in the art that they are used, as exemplified by a variety of technical dictionaries. Where a term is provided in the singular, the inventors also contemplate the plural of that term. The nomenclature used herein and the procedures described below are those well known and commonly employed in the art. Where there are discrepancies in terms and definitions used in references that are incorporated by reference, the terms used in this application shall have the definitions given herein. Other technical terms used herein have their ordinary meaning in the art that they are used, as exemplified by a variety of technical dictionaries (for example, Chambers Dictionary of Science and Technology, Peter M. B. Walker (editor), Chambers Harrap Publishers, Ltd., Edinburgh, UK, 1999, 1325 pp.). The inventors do not intend to be limited to a mechanism or mode of action. Reference thereto is provided for illustrative purposes only.

I. Method for Enhancing Degradation of a Transcription Factor

The present invention includes a method of enhancing or increasing the degradation of a nuclear receptor (NR) or STAT transcription factor protein. The methods of the present invention may therefore inhibit a nuclear receptor activation pathway or STAT activation pathway or both. The methods may include providing a cell comprising a nuclear receptor pathway or STAT activation pathway and introducing a compound capable of enhancing degradation of a nuclear receptor or a STAT transcription factor protein.

Nuclear receptors (NR) are transcription factors that activate transcription of target genes in response to ligand. NRs are typically divided into type I, type II and type III. Type I nuclear receptors include the classical steroid receptors (androgen receptor (AR), estrogen receptor α and β (ER), progesterone receptor (PR), glucocorticoid receptor (GR), and mineralocortocoid receptor (MR). Type II nuclear receptors dimerize with the 9-cis retinoic acid receptor (RXR) and include the receptors for vitamin D (VDR), thryroid hormone (TR), trans-retinoic acid receptor (RAR), and the peroxisome proliferator-activated receptors (PPAR). Type III includes orphan nuclear receptors (ONRs).

The method of the present invention may be applied to any one or more nuclear receptors or transcription factors of interest, where degradation of the nuclear receptor is desired. Transcription factors of interest include the above-referenced type I, type II and type III nuclear receptors, steroid hormone receptors, thyroid hormone receptor-like factors, STAT transcription factors, heat shock factors, and the like. A list of suitable transcription factors is provided, for example, by the eukaryotic transcription factor database TRANSFAC® available at www.gene-regulation.com/pub/databases/transfac/c1.html (accessed 27 Aug. 2004), which is incorporated by reference in its entirety herein. Nuclear receptors of special interest include steroid hormone receptors, such as androgen receptors, progesterone receptors, estrogen receptors (α and β), glucocorticoid receptors, peroxisome proliferator-activated receptors (PPAR), 9-cis retinoic acid receptors (RXR), all trans-retinoic acid receptors (RAR) and orphan steroid hormone receptors.

Most retinoid forms activate RAR family members, whereas RXR family members are activated by 9-cis-Retinoic acid only. RAR family members, which include RARα, RARβ and RARγ have a high affinity for all trans-retinoic acids and belong to the same class of nuclear transcription factors as thyroid hormone receptors, vitamin D3 receptor and ecdysone receptor. The human RARα gene maps to chromosome 17.

The method may be applied to non-cell based screens, such as assays using cell-free preparations or isolated nuclear receptors, to isolated cells, such as cells grown in in vitro culture for assay purposes, or to intact, living subjects. Suitable subjects include mammals of research, agricultural, or economic interest, including rodents, lagomorphs, canids, felids, swine, bovids, and non-human primates. Subjects can include human subjects of any age, sex, or physical condition. Subjects of particular interest include human subjects diagnosed as having, or as being at risk for, a disease condition or conditions that are, at least in part, affected by the activity of a nuclear receptor such as a steroid hormone receptor (for example, an androgen receptor, a progesterone receptor, or an estrogen receptor). Such disease conditions include, but are not limited to, cancers (for example, prostate cancer, liver cancer, bladder cancer, and other cancers which involve the androgen receptor activation pathway, and breast cancer, which is affected by the androgen receptor and estrogen receptor), neurological and neuromuscular disorders (for example, Kennedy Disease, which is affected by the androgen receptor), skin disorders (for example, acne, which is caused by androgen-induced AR activation of sebaceous glands), hair disorders (for example, androgenetic alopecia or “male pattern baldness”, where hair loss is caused in part by the androgen receptors in follicles and adjacent cells), and wound healing (where inflammation is affected by the androgen receptor in response to androgen). Subjects may also include female animals in which pregnancy is not desired, or women of child-bearing age who do not wish to become pregnant, where degradation of the progesterone receptor may be useful in preventing conception or in inducing still birth.

The method may include administering one or more compounds able to enhance, induce or increase the rate of degradation of a nuclear receptor (NR), such as, but not limited to, a steroid hormone receptor. The one or more compounds may act using one or more mechanisms of nuclear receptor degradation. Preferably, administering the one or more compounds results in the specific degradation of a targeted nuclear receptor, without substantially altering the levels or activity of other, non-targeted transcription factors or nuclear receptors.

It may be desirable to induce or enhance degradation of a targeted nuclear receptor or STAT transcription factor protein to a different degree or in a targeted tissue or cell type. For example, as different tissues or cell types are responsive to a given steroid to different degrees, it may be desirable to induce or enhance degradation of the appropriate steroid hormone receptor in one tissue or cell type but not another, or it may be desirable to lower the targeted steroid hormone receptor to different “thresholds” or levels according to the tissue or cell type. In some embodiments, the method may include administering the one or more compounds in a quantity sufficient to degrade the targeted nuclear receptor in a given tissue or cell type, thus lowering the targeted nuclear receptor to a desired level (for example, where the targeted nuclear receptor is the androgen receptor, the desired androgen receptor level may be a level that is substantially non-responsive to circulating androgen).

The method of the invention may make use of any one or more suitable mechanisms to induce, increase or enhance the degradation of a nuclear receptor. These mechanisms include, but are not limited to, interfering with translocation of the nuclear receptor into the nucleus or retaining the nuclear receptor in the cytoplasm of a cell, exposing a motif within the nuclear receptor able to induce protease activity, increasing activity of a protease capable of degrading the nuclear receptor, inhibiting the stabilization of a nuclear receptor, reducing the solubility of the nuclear receptor, activating a pathway able to degrade the nuclear receptor, increasing ubiquination of the nuclear receptor, increasing phosphorylation of the nuclear receptor by an appropriate kinase (for example, in the case of the androgen receptor, by activating Akt kinase, which in at least some cases phosphorylates the androgen receptor, leading to the receptor's ubiquination and subsequent degradation by the proteosome; see, for example, Heinlein and Chang (2004) and Lin et al. (2002) EMBO J., 21:4037-4048, which are incorporated by reference in their entirety herein), inducing apoptosis, or reducing an interaction between a nuclear receptor and a cofactor able to stabilize the nuclear receptor.

In one embodiment, the method of the present invention induces or enhances degradation of a nuclear receptor by interfering with the translocation of the nuclear receptor into the nucleus. For example, androgen-bound androgen receptors are translocated from the cytoplasm to the nucleus where they regulate genes using a zinc finger motif. When blocked from translocation or when retained within the cytoplasm, the androgen receptor undergoes proteolysis, and is thus unable to affect the nuclear DNA.

In another embodiment, the method of the present invention enhances or induces degradation of a nuclear receptor by exposing within the nuclear receptor a site or motif that is able to induce proteolysis in the presence of a protease. Such exposure may occur by inducing a conformational change of a domain within the nuclear receptor, such as by binding a compound to the nuclear receptor or by phosphorylating a domain of the nuclear receptor. For example, in the case of the androgen receptor, a PEST (proline-, glutamate-, serine-, and threonine-rich) motif has been identified as able to induce ubiquitin-dependent protoeolysis in the presence of the E3 ligase Mdm2 and has been found in a hinge region of the androgen receptor (Lin et al. (2002) EMBO J., 21:4037-4048, which is incorporated by reference in its entirety herein). A compound able to expose a motif such as PEST in the presence of a ligase such as Mdm2 may induce or enhance degradation of the androgen receptor and thus inhibit the androgen receptor-activated pathway. In an alternative embodiment, increasing the activity of a suitable nuclear receptor-specific protease may induce or enhance the increase in nuclear receptor degradation.

In another embodiment, the method of the present invention induces or enhances degradation of a nuclear receptor by preventing or reducing stabilization of the nuclear receptor. Preventing or decreasing stabilization may occur by inhibiting interactions between two or more nuclear receptor or by preventing or decreasing the interaction between a nuclear receptor and a cofactor. In one example, the androgen receptor is believed to dimerize with another androgen receptor, thereby increases the stability of the receptor. Dimerization may occur via interaction between the amino terminals of the receptors. Preventing dimerization, for example, by administering a compound able to reduce or eliminate dimerization by binding at or near the amino terminal domain, may induce or enhance the degradation of the androgen receptor and thus inhibit the androgen receptor activated pathway. In another embodiment, the interaction between a nuclear receptor and a cofactor may be disrupted, thereby inducing degradation of the nuclear receptor. In one embodiment the present invention disrupts the interaction between a nuclear receptor and a STAT (signal transducer and activator of transcription) protein.

In another embodiment, the method of the present invention induces or enhances degradation of a nuclear receptor by inhibiting or reducing the binding of a stabilizing cofactor to the nuclear receptor. For example, steroid receptor coactivator 1 (SRC-1) is a cofactor believed to bind to the amino terminal of the androgen receptor and the DNA binding domain (DBD). A compound able to inhibit or reduce the binding of SRC-1, such as by directly or indirectly blocking the binding site between SRC-1 and the androgen receptor, or by competing for binding with either SRC-1 or the androgen receptor, may prevent or reduce SRC-1 stabilization of the androgen receptor and thus result in increased degradation of the androgen receptor.

In another embodiment, the method of the present invention induces or enhances degradation of a nuclear receptor by destabilization of a domain of the nuclear receptor. For example, some studies suggest that the AF-2 domain of the androgen receptor stabilizes the receptor's overall structure, allowing the amino terminal domain to interact with coregulators. A compound able to destabilize the AF-2 domain, for example, by interacting or binding with the AF-2 domain or with a domain that itself interacts with the AF-2 domain, may reduce the stabilization of the amino terminal domain, reduce the interaction with coregulators, and increase the rate of degradation of the androgen receptor.

In another embodiment, the method of the present invention induces or enhances degradation of a nuclear receptor by activating a pathway able to degrade the nuclear receptor. For example, the caspase-3 pathway has been suggested to induce degradation of the androgen receptor (Lee and Chang (2003)). Activation of the caspase-3 pathway may occur by the presence of a tumor suppressor, phosphatase and tensin homologue, PTEN, such as by an interaction between the AR DNA-binding domain and the PTEN phosphatase domain, believed to lead to AR retention in the cytoplasm. Therefore a compound able to induce or enhance the caspase-3 pathway may induce or enhance the degradation of the androgen receptor. In another example, Akt kinase, which phosphorylates the androgen receptor, may be activated, leading to ubiquination and subsequent degradation of the androgen receptor by the proteosome (Heinlein and Chang 2004).

In some aspects of the invention, interactions of STAT are reduced or inhibited. The methods may include but are not limited to inhibiting the phosphorylation of STAT, reducing or inhibiting dimerization of STAT, reducing or inhibiting binding between STAT and a nuclear receptor and the like. Thus the present invention includes methods of targeting or reducing the STAT signalling Pathway.

STAT (signal transducer and activator of transcription) proteins are a family of latent cytoplasmic transcription factors involved in cytokine, hormone nuclear receptor, and growth factor signal transduction. STAT transcription factor proteins mediate broadly diverse biologic processes, including cell growth, differentiation, apoptosis, fetal development, transformation, inflammation, and immune response. Seven members of the STAT family of transcription factors have been identified in mammalian cells: STAT1, STAT2, STAT3, STAT4, STAT5a, STAT5b, and STAT6.

At least some of the STAT transcription factor proteins are activated by the Janus superfamily tyrosine kinases (JAKs), which themselves are activated by interferons, IL-1, IL-2, IL-3, IL-4, IL-5, IL-6, IL-7, IL-11, IL-12, IL-13, growth hormone (GH), prolactin (PRL), erythropoietin (EPO), thrompopoietin, and tumor necrosis factor (TNF), granulocyte-macrophage colony-stimulating factor (GM-CSF). STATs are believed to be phosphorylated on a single tyrosine residue in the carboxy terminal portion. The modified STATs form homodimers or heterodimers through reciprocal interaction between the phosphotyrosine of one STAT and the SH2 domain of another. Following dimerization, STATs, like nuclear receptors, are translocated to the nucleus and interact with specific regulatory elements to induce or enhance target gene transcription. By interfering with the dimerization of a STAT transcription factor protein or interfering with the phosphorylation of STAT by JAK, the methods of the present invention may reduce the effect of STAT or enhance or induce degradation of STAT.

STAT1 is believed to be involved in the TNFR1-TRADD signalling complex. In Hela cells STAT1 acts as a TNFR1-signaling molecule to suppress NF-kB activation. It has also been shown that its activation slows growth and promotes apoptosis. After phosphorylation, STAT1 and STAT2 form heterodimers that function as more potent inducers of transcription than the STAT1 homodimer. STAT1 is known to associate with the transcription factors p48, Sp1, and p300. Human STAT2 cDNA codes for a 851 amino acid protein with a predicted molecular weight of 113 kDa. STAT2 associates with beta subunit of the type I IFN receptor in an interferon-dependent manner. The unique acidic domain of the carboxy-terminal region of STAT2 may interact with cAMP-response-element binding protein. STAT3 has been shown to be activated by IFN-alpha but not IFN-beta. The transcription factors associated with STAT3 are c-Jun and cyclic AMP-responsive enhancer binding protein (CREB). STAT4 is activated when cells are treated with interleukin-12, a key cytokine regulator of cell-mediated immunity. STAT4 is also activated by IFN alpha/beta. In STAT4 knock-out mice, lymphocytes no longer proliferate in response to IL-12, produce IFN gamma, or express natural killer cell cytotoxicity. Both STAT5a and STAT5b regulate interleukin-7 induced B-cell precursor expansion. STAT5b may also act as a transcriptional inhibitor as demonstrated by inhibition of NF-kB mediated signaling. This STAT5b-mediated inhibitory effect on NF-kB signaling does not depend on STAT5b-DNA interactions but requires the carboxyl terminus of STAT5b as well as STAT5b nuclear translocation and/or accumulation, suggesting that STAT5b is competing for a nuclear factor(s) necessary for NF-kB-mediated activation of target promoters. STAT5 has been theorized to be the physiological substrate of the insulin receptor. STAT5 has been shown to be the essential mediator of prolactin induced milk protein gene activation. STAT6 has been shown to be activated by interleukin-4 (IL-4), IL-13, and IL-3. In STAT6 deficient knock-out mice, B lymphocytes fail to proliferate and mature in response to IL-4 and T-Lymphocytes differentiation and proliferation is impaired. STAT6 is rapidly tyrosine phosphorylated following stimulation of appropriate cell lines with IL-3, IL-4, epidermal growth factor (EGF), but is not detectably phosphorylated following stimulation with IL-2, IL-12, or erythropoietin.

Inhibiting or reducing the effect of STAT may allow the selective inhibition of a variety of molecules involved in the immune response or nuclear receptor associated disorders. For example, the present invention may counter act in part the stimulatory effect of IL-1 (α and β), IL-2, IL-3, IL-4, IL-5, IL-6, IL-7, IL-11, IL-12, IL-13, growth hormone (GH), prolactin (PRL), erythropoietin (EPO), thrompopoietin, and granulocyte-macrophage colony-stimulating factor (GM-CSF), epidermal growth factor (EGF), NF-kB, tumor necrosis factore (TNF) and the like. The present application will have particular utility in the prevention of inflammation which occurs via stimulation by cytokines or other stimuli such as but not limited to exposure to a microbe, microbial sample or antigen.

II. Compositions for Inducing Degradation of a Transcription Factor

The present invention also includes a pharmaceutical or cosmetic composition capable of inducing degradation of a transcription factor, nuclear receptor or STAT transcription factor protein and thereby preventing or treating causes or symptoms of disease conditions that are, at least in part, affected by the activity of the transcription factor or nuclear receptor. Transcription factors of interest include steroid hormone receptors, thyroid hormone receptor-like factors, STAT transcription factors, heat shock factors, and the like. Transcription factors of especial interest include steroid hormone receptors, such as androgen receptors, progesterone receptors, estrogen receptors (α and β), glucocorticoid receptors, peroxisome proliferator-activated receptor (PPAR), 9-cis retinoic acid receptors (RXR), trans-retinoic acid receptors (RAR), and orphan steroid hormone receptors. Steroid hormone receptors of particular interest include, but are not limited to, the androgen receptor and the progesterone receptor. Compositions of particular interest are those capable of enhancing or inducing specific degradation of a steroid hormone receptor, such as, but not limited to, the androgen receptor, estrogen receptor, or progesterone receptor. The compositions of the invention may affect their activity by means of any one or more suitable mechanisms to induce or enhance the degradation of a transcription factor. Suitable mechanisms, and non-limiting embodiments illustrating these mechanisms, are described in detail above in the section headed, “I. METHOD FOR ENHANCING DEGRADATION OF A TRANSCRIPTION FACTOR”.

The compositions of the invention may include one or more active compounds or effective variations thereof in a suitable carrier. Any suitable active compounds may be of use, if they are capable of enhancing or inducing degradation of the transcription factor of interest. Preferably, these compounds are effective at enhancing or inducing degradation of the transcription factor of interest at physiologically acceptable levels, such as at levels that do not cause undesirable side effects or toxicity. The active compound can optionally include one or more elements that provide additional benefits, such as improved stability, solubility, or delivery specificity. Such elements can include peptides, polypeptides, proteins, carbohydrates, nucleic acids, lipophilic moieties, hydrophilic moieties, particulates, matrices, or combinations thereof. For example, the active compound can be linked, covalently or non-covalently, to a hydrophilic moiety (such as a phosphate or sulphate group or a carbohydrate or a chelating molecule) to improve solubility in aqueous buffers or bodily fluids. In another example, the active compound or active fragment thereof may be treated to reduce toxicity or cytotoxicity. In another example, the active compound can be linked, covalently or non-covalently, to a peptide or other moiety that protects the active compound from premature degradation, or to an antibody or other specific binding agent that specifically targets a desired tissue or cell type and thus improves delivery of the active compound to that specific tissue or cell type. In another example, the active compound can be encapsulated or embedded in a liposome, a particulate, a matrix, a gel, a polymer, or the like, to improve stability or to enhance delivery.

Nonlimiting examples of suitable compounds are curcumin derivatives or analogues thereof capable of enhancing degradation of the desired nuclear receptor or STAT transcription factor protein. A number of curcumin analogues have been identified in U.S. Pat. No. 6,790,979, which is herein incorporated by reference in its entirety. As shown in the examples and brief description of the figures, the curcumin analogues JC9 and JC15 are effective at enhancing degradation of a nuclear receptor when provided at pharmaceutically acceptable levels.

Suitable carriers of use in the compositions of the invention include diluents, excipients, or carrier materials, selected according to the intended form of administration and consistent with conventional pharmaceutical or cosmetic practice. Examples of suitable carriers include, but are not limited to, water, physiological saline, phosphate-buffered saline, a physiologically compatible buffer, saline buffered with a physiologically compatible salt, a water-in-oil emulsion, and an oil-in-water emulsion, an alcohol, dimethylsulfoxide, dextrose, mannitol, lactose, glycerin, propylene glycol, polyethylene glycol, polyvinylpyrrolidone, lecithin, albumin, sodium glutamate, cysteine hydrochloride, and the like, and mixtures thereof. Suitable carriers can also include appropriate pharmaceutically acceptable antioxidants or reducing agents, preservatives, suspending agents, solubilizers, stabilizers, chelating agents, complexing agents, viscomodulators, disintegrating agents, binders, flavoring agents, coloring agents, odorants, opacifiers, wetting agents, pH buffering agents, and mixtures thereof, as is consistent with conventional pharmaceutical practice (“Remington: The Science and Practice of Pharmacy”, 20th edition, Gennaro (ed.) and Gennaro, Lippincott, Williams & Wilkins, 2000).

For use in isolated cells, such as in cells grown in culture and used in bioassays, compositions of the present invention can be formulated and provided as is convenient. In non-limiting examples, compositions may be formulated as dissolvable solids, solutions, suspension, liposome preparations, and the like, and provided to the cells by manual or automated delivery (such as by pipette, syringe, pump, auto-injector, and the like).

For use in a living, whole organism, such as in a human subject, compositions of the present invention can be formulated and provided in any formulation suitable to the intended form of administration and consistent with conventional pharmaceutical practice (“Remington: The Science and Practice of Pharmacy”, 20^{th} edition, Gennaro (ed.) and Gennaro, Lippincott, Williams & Wilkins, 2000). Examples of suitable formulations include tablets, capsules, syrups, elixirs, ointments, creams, lotions, sprays, aerosols, inhalants, solids, powders, particulates, gels, suppositories, concentrates, emulsions, liposomes, microspheres, dissolvable matrices, sterile solutions, suspensions, or injectables, and the like. Injectables can be prepared in conventional forms either as liquid solutions or suspensions, as concentrates or solid forms suitable for solution or suspension in liquid prior to injection, or as emulsions.

For use in a living, whole organism, such as in an animal or in a human subject, and depending on the specific conditions being treated, pharmaceutical compositions of the present invention can be formulated and administered systemically or locally. Techniques for formulation and administration can be found in “Remington: The Science and Practice of Pharmacy” (20^{th} edition, Gennaro (ed.) and Gennaro, Lippincott, Williams & Wilkins, 2000). Suitable routes of administration can include oral, intestinal, parenteral, transmucosal, transdermal, intramuscular, subcutaneous, transdermal, rectal, intramedullary, intrathecal, intravenous, intraventricular, intraatrial, intraaortal, intraarterial, or intraperitoneal administration. The pharmaceutical compositions of the present invention can be administered to the subject by a medical device, such as, but not limited to, implantable devices, biodegradable implants, patches, and pumps. Where such a device is used, the compositions may be formulated to include a dissolvable or nondissolvable matrix or medium (for example, a coating, membrane, film, impregnated matrix, polymer, sponge, gel, or porous layer on or within the medical device) to permit the release of the active compound or compounds over a specified period of time.

III. Method for Treating Transcription Factor-Associated Diseases

The present invention also includes methods of treatment of a variety of transcription factor-associated diseases or conditions by inducing the degradation of a transcription factor such as a nuclear receptor. Diseases or conditions of interest include any that are associated with a specific transcription factor or nuclear receptor, and that have causes or symptoms that may be prevented, reduced, or reversed by degradation of the transcription factor or nuclear receptor in question. Transcription factors of interest include steroid hormone receptors, thyroid hormone receptor-like factors, STAT transcription factors, heat shock factors, and the like. Transcription factors of especial interest include steroid hormone receptors, such as androgen receptors (AR), progesterone receptors (PR), estrogen receptors (ER α and ER β), glucocorticoid receptors (GR), peroxisome proliferator-activated receptor (PPAR), retinoid X receptors (RXR), retinoid A receptors (RAR) and orphan steroid hormone receptors. Steroid hormone receptors of particular interest include, but are not limited to, the androgen receptor and the progesterone receptor.

Any suitable subject may be treated by the method of the present invention. Subjects can include human subjects of any age, sex, or physical condition. Subjects of particular interest include human subjects diagnosed as having, or as being at risk for, a disease condition or conditions that are, at least in part, affected by the activity of a nuclear receptor (such as, but not limited to, a steroid hormone receptor). In one embodiment, subjects may be humans having or at risk of developing a disease, disorder or medical condition associated with expression or activity of a nuclear receptor. For example, subjects may be humans having or at risk of developing a disease or disorder associated with the activity of a nuclear receptor such as the androgen receptor (AR). Examples of medical conditions or diseases include, but not limited to, male infertility, Kennedy disease, prostate cancer, breast cancer, liver cancer, bladder cancer, benign prostate hyperplasia, acne, baldness, and hirsutism. The medical condition may result from increased activity or expression of the nuclear receptor pathway or may be due to a mutation in the nuclear receptor activation pathway. For example, Kennedy disease is caused by an abnormally elongated androgen receptor. Modulation of AR function or activity by enhancing or inducing AR degradation by a method of the present invention may serve as a means to prevent the development and progression of prostate cancer and to remedy other androgen-related disorders. Use of the method of the present invention to enhance or induce or induce degradation of the androgen receptor may also have utility in the treatment of wounds, such as by improving wound healing, especially in aging men or a diabetic patient. In another embodiment, the method of the invention can be used to enhance or induce degradation of the progesterone receptor to prevent conception in females (such as women of child-bearing age).

The compositions, cosmetics, pharmaceuticals and methods of the present invention may be utilized to prevent or treat a variety of skin disorders or other medical conditions. For example, the present invention may be useful in the treatment or prevention of Acne, Acne keloidalis nuchae, Acne necrotica, Acne urticata, Acne Vulgaris, Actinic Keratoses, Acute Febrile Neutrophilic Dermatosis, Aging skin, Allergic contact dermatitis, Alopecia greata, Androgenetic alopecia, Angioedema, Anthralin, Atopic dermatitis, Bacterial skin infections, Boils, Botulinum toxin, Bowen's disease, Brown spots, Chronic superficial scaly dermatosis, Cysts, Dermatitis, Dermatitis herpetiformis, Dermatofibromas, Dermographism, Eczema, Eczema Vaccinatum, Erythrodermic Psoriasis, Facial Rejuvenation, Folliculitis, Guttate Psoriasis, Hair loss, Halo moles, Hand dermatitis, Male pattern alopecia, Parapsoriasis (small plaque), Psoriasis, Psoriasis vulgaris, Shingles, Skin cancer, Staphylococcal scalded skin syndrome, Staphylococcal skin infections, Steatocystoma multiplex, Steroid acne, Steroid rosacea, Streptococcal infections, Sun protection, or Viral skin conditions.

The nuclear receptor of interest can be a normal nuclear receptor or a mutant nuclear receptor, where the mutation may be associated with disease symptoms or causes. For example, Kennedy Disease is one of a group of hereditary neuropathies (including Huntington's disease, spinocerebellar ataxia types 1, 6, and 7, Machado-Joseph disease, and dentatorubropallidoluysian atrophy) with a genetic origin in an expanded area of translated CAG trinucleotide repeats in the androgen receptor, which produces abnormally long polyglutamine stretches within the respective gene products. Of these neuropathies, the affected nuclear receptor gene product (the androgen receptor, AR) has been characterized only for Kennedy Disease. In Kennedy Disease, there is a CAG expansion in exon 1 of the AR gene (see Bailey et al. (2002) Human Mol. Genetics, 11:515-523, which is incorporated by reference in its entirety herein). Studies indicate that the polyglutamine-expanded AR is structurally altered so as to be resistant to proteolysis, and may give rise to a possibly post-translationally misprocessed, denaturation- and proteolysis-resistant fragment that accumulates in the nucleus of the affected cells and may cause the observed pathogenic effects (Abdullah et al. (1998) Human Mol. Genetics, 7:379-384, which is incorporated by reference in its entirety herein). Short polyglutamine repeats have also been implicated in an increased risk of developing prostate cancer (Lee and Chang (2003)), and may be associated with an increased risk of infertility (defective spermatogenesis) and undermasculinization in at least some male populations (Yong et al. (2003) Hum. Reprod. Update, 9(1): 1-7).

Like other polyglutamine diseases, Kennedy Disease is characterized by neuronal intranuclear inclusions in the affected neurons. These inclusions contain an N-terminus truncated form of the mutant nuclear receptor (the androgen receptor, AR), and are associated with ubiquitin and proteosome components, suggesting that the polyglutamine-expanded AR is not adequately degraded by the proteosome and accumulates in the inclusions as a result (Bailey et al. (2002) Human Mol. Genetics, 11:515-523). In a cell culture model of Kennedy disease, chaperone proteins have been shown to increase the solubility of the mutant AR and thus enhance its degradation by the proteosome (Bailey et al. (2002) Human Mol. Genetics, 11:515-523). Enhancing or inducing degradation of the mutant nuclear receptor (the androgen receptor) by a method of the present invention may be useful in preventing or reducing the pathogenic effects associated with the mutant nuclear receptor. Such degradation can be accomplished by any suitable mechanism, such as, but not limited to, repair of the mutant AR to allow correct degradation, molecular chaperone enhancement of mutant AR solubilization and subsequent degradation, and prevention of overexpressed AR from forming aggregates. This approach may have therapeutic value in other polyglutamine diseases characterized by the accumulation of neuronal intranuclear inclusions.

It has been reported that, among the elderly, males heal acute wounds more slowly that do females. Elderly males also have an altered inflammatory response, and recent studies indicate that testosterone, via the androgen receptor pathway, directly upregulates TNF-alpha and thus the inflammatory response (Ashcroft and Mills (2002) J. Clin. Invest., 110:615-624, which is incorporated by reference in its entirety herein). AR was also found to be expressed not only within normal skin but also in acute wounds, including epithelial cells, hair follicles, fibroblasts, and macrophages. These studies further demonstrate that accelerated wound healing, associated with increased collagen matrix deposition and a dampened inflammatory response, can be achieved by either castration or by directly blocking the AR pathway. Enhancing or inducing degradation of the androgen receptor utilizing methods of the present invention may be useful in accelerating or enhancing wound healing in males, especially elderly males, or in diabetic or ulcer patients.

Any suitable mechanism may be used to induce or enhance the degradation of a nuclear receptor. Suitable mechanisms, and non-limiting embodiments illustrating these mechanisms, are described in detail above in the section headed, “I. METHOD FOR ENHANCING DEGRADATION OF A TRANSCRIPTION FACTOR”.

### EXAMPLES

### Example 1

### Overview of Enhancing Degradation of a Transcription Factor

The following examples describe non-limiting embodiments of a method of enhancing degradation of a nuclear receptor. Any mechanism that enhances degradation of the nuclear receptor of interest can be used, including, but not limited to, interfering with translocation of the nuclear receptor into the nucleus or retaining the nuclear receptor in the cytoplasm of a cell, exposing a motif within the nuclear receptor able to induce protease activity, increasing activity of a protease capable of specifically degrading the nuclear receptor, inhibiting the stabilization of a nuclear receptor, reducing the solubility of the nuclear receptor, activating a pathway able to degrade the nuclear receptor, increasing ubiquination of the nuclear receptor, increasing phosphorylation of the nuclear receptor by an appropriate kinase, inducing apoptosis, or reducing an interaction between a nuclear receptor and a cofactor able to stabilize the nuclear receptor. In this particular example, the nuclear receptor of interest is the steroid hormone receptor, the androgen receptor.

Various methods and assays can be used to detect down regulation of nuclear receptor transcriptional activity and therefore degradation of the nuclear receptor of interest, or to detect the downstream effects of such degradation. For example, assays used to detect the down regulation of the androgen receptor may be used at least in part to detect degradation of the androgen receptor. Non-limiting examples of such methods and assays, as applicable to the androgen receptor, are described in general below.

Detection of AR Degradation Using Western Blot Analysis

A Western blot method suitable for detecting degradation of the androgen receptor (AR) has been previously described (Su et al., 1999). Briefly, cells (for example, LNCap cells) are harvested either in 2× sodium dodecyl sulfate (SDS) loading buffer or in radioimmunoprecipitation assay (RIPA) lysis buffer (see “Antibodies: A Laboratory Manual”, E. Harlow and D. Lane, Cold Spring Harbor Laboratory, 1988) containing 10 micrograms/milliliter of benzamidine, 10 micrograms/milliliter of trypsin inhibitor, and 1 millimolar of phenylmethylsulfonyl fluoride (PMSF). Total protein (40 micrograms/sample, or as desired) from cell lysate is separated on a SDS-PAGE gel. After separation, proteins are transferred from the gel to a nitrocellulose membrane following standard Western blot procedures. The membrane is blocked with a suitable blocking agent (such as 10% non-fat milk in phosphate-buffered saline supplemented with 0.1% Tween-20 (PBST)) overnight. The membrane is incubated with a suitable primary antibody specific for human AR (for example, anti-human AR from BD-PharMingen) at 4 degrees Celsius overnight or at room temperature for 2 hours. The membrane is rinsed with PBST three times, 10 minutes each time, and then incubated with an appropriate secondary antibody (for example, an enzyme-labelled secondary antibody, such as horseradish peroxidase-conjugated secondary antibody) for 1 hour at room temperature. The membrane is rinsed with PBST, and a suitable visualization procedure is used to detect the secondary antibody (for example, horseradish peroxidase can be detected with a colorimetric substrate or by a chemiluminescent substrate such as that provided by the enhanced chemiluminescence (ECL Plus) kit from Amersham). The secondary antibody signal, as a measure of the amount of androgen receptor protein on the blot, can be normalized to the total amount of protein loaded for each sample by stripping the membrane following the manufacture's recommendations and re-incubating the membrane with an appropriate antibody (such as an antibody to beta-actin, Sigma). Quantification of the protein signals can be carried out by densitometry, using appropriate software (ImageJ software from the National Institutes of Health).

Compound that Inhibits AR Activity and Tumor Cell Growth

In the non-limiting examples, compounds were used to degrade the androgen receptor (AR) in cells. Non-limiting examples of compounds that were tested for their ability to degrade AR include curcumin derivatives and analogues, whose structures and preparations are described in Ohtsu et al. (2002), J. Med. Chem., 45:5037-5042 and Ohtsu et al. (2003) Bioorg. Med. Chem., 11:5083-5090, which are incorporated by reference in their entirety herein. As examples, the compounds JC15 and JC9 were tested on cultured cells. JC15 is a synthetic curcumin derivative bearing an ethoxycarbonylethyl moiety on carbon 4 of the conjugated beta-diketone system and has the structure 7-(4-hydroxy-3-methoxyphenyl)-4-[3-(4-hydroxy-3-methoxyphenyl)acryloyl]-5-oxo-hept-6-enoic acid ethyl ester. JC9 is another synthetic curcumin derivative (trivial name, dimethylcurcumin), and has the structure 5-hydroxy-1,7-bis(3,4-dimethoxyphenyl)-1,4,6-heptatrien-3-one; it is obtainable, for example, by permethylation of natural curcumin with diazomethane.

### Example 2

### Enhancing Degradation of a Nuclear Receptor

This describes a non-limiting example of methods and assays useful in studying the downstream effects of degradation of a nuclear receptor. In this particular example, a compound known to enhance degradation of the nuclear receptor, androgen receptor (AR), was examined for its effects on AR activity and on cell proliferation. One of the major tasks in cancer management is to control or slow tumor proliferation. AR plays a significant role in stimulating prostate cancer cell proliferation, and thus, modulation of AR activity by AR degradation could serve as a useful means to delay or control prostate cancer progression.

Androgen Receptor Transactivation Assay

Prostate cancer cells, non-prostate tumor cells, and normal cells can be used in this transient transfection assay, which measures transactivation of the androgen receptor (AR) and can be used to detect a reduction of AR activity caused by AR degradation. In the provided non-limiting example, CV-1 (green monkey kidney cells lacking an androgen receptor) and LNCaP cells (prostate cancer cells containing an endogenous mutant AR), were transfected with a plasmid carrying an androgen response element (ARE) and a reporter gene.

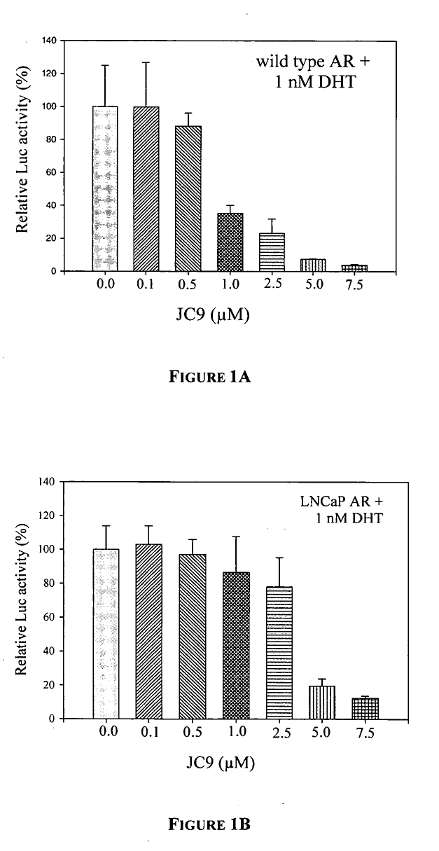

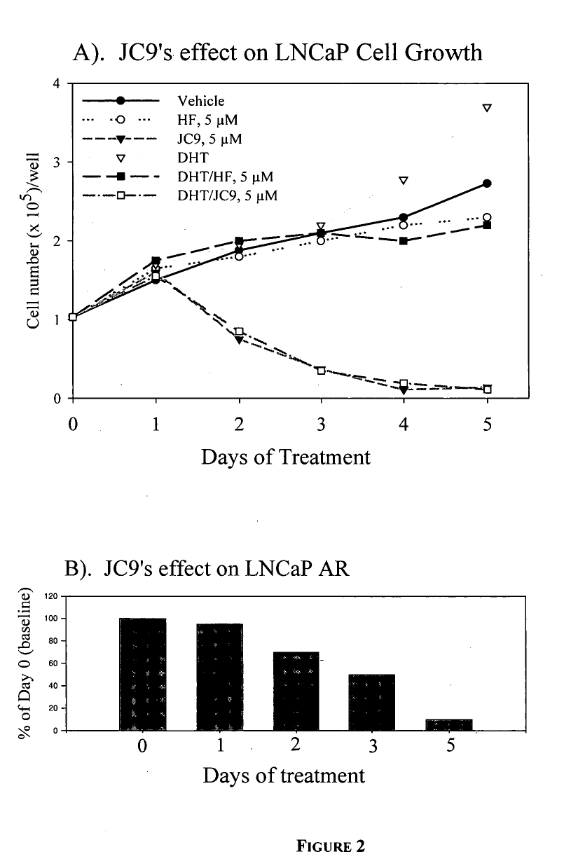

Both the CV-1 and LNCaP cells (obtained from ATCC, Manassas, Va.) were maintained in Richter's Improved MEM Insulin (RPMI) medium supplemented with penicillin (25 units/millilter), streptomycin (25 micrograms/milliliter), and 10% heat-inactivated fetal bovine serum (FBS). Cells were plated in 35-millimeter dishes at a concentration of 1-2×10⁵cells/well and cultured at 37 degrees Celsius in an incubator under a 5% carbon dioxide atmosphere. One day later, the cells were co-transfected with the SuperFect Transfection Reagent (Qiagen) and DNA mixtures consisting of an MMTV-luciferase reporter plasmid containing an AR-binding element. The plasmid pRL-TK was used as an internal control for transfection efficiency. After a 3-hour incubation, the medium was changed to RPMI medium supplemented with 10% charcoal-stripped serum. Twenty hours later, the cells were treated with the test compound at varying concentrations (from 0 uM to 7.5 uM), in the presence or absence of 1 nanomolar dihydrotestosterone (DHT), and incubated for a further 20 hours. The cells were lysed, and luciferase activity of the lysate was measured with the Dual-Luciferase Reporter Assay System (Promega, Madison, Wis.). Data were expressed as relative luciferase activity normalized to the internal luciferase positive control. Results are depicted in FIG. 1A and FIG. 1B. In summary, JC9 was capable of inhibiting DHT-induced androgen receptor transcriptional activation in a does dependent manner.

Detection of Cell Growth and Androgen Receptor Expression in LNCaP Cells

Growth Assay

LNCaP cells express an endogenous mutant AR that is found in prostate cancer patients. This clinically relevant cell model was used to study JC9's effect in suppressing prostate cancer cell growth. Cells were plated at a density of 6.3×10⁴ cells/well in 6-well tissue culture dishes. Two days later, the complete medium was aspired and 10% charcoal/dextran-treated (hormone depleted) serum-containing medium was added. Test compounds, JC9 and hydroxyflutamide (HF) were then added to the medium at a final concentration of 5 μM with or without 1 nM of DHT. For vehicle control, the same amount of DMSO was added. For the subsequent 5 days, the medium was aspired once per day, and fresh medium containing test compounds and/or DHT was added. At designated times, a portion of cells was harvested by trypsinization, and cell count was performed using a hemacytometer.

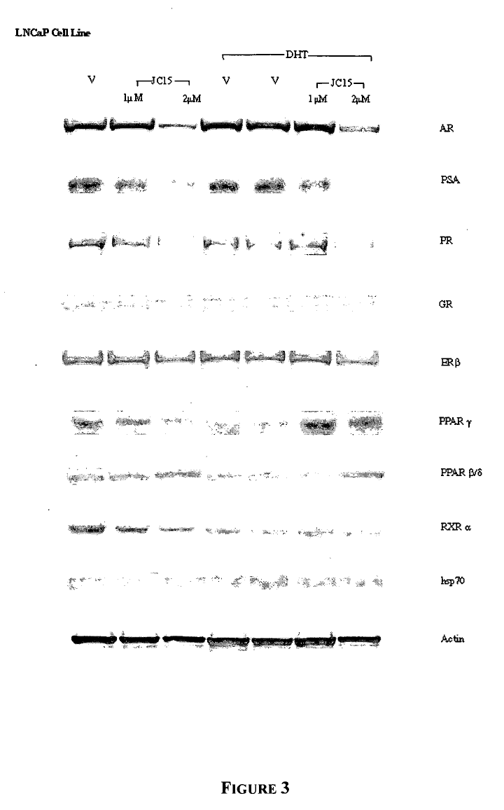

During a 5-day experimental duration, the cell number in vehicle control-treated wells steadily increased (FIG. 2A). On Day 1, the cell number of JC9-treated wells was comparable to that of vehicle control, but starting from Day 2, there was a marked decrease in cell number in these wells. At the end of a 5-day incubation, only a minimal number of viable cells were found in the JC9-treated wells. This dramatic growth suppressing effect, however, was not detected in HF-treated dishes.

JC9's effect on prostate cancer cell growth was further assessed in the presence of a prostatic androgen, DHT. As expected, DHT up-regulated LNCaP cell growth; a raise in cell number per well became visible after the cells were allowed to be incubated with this male hormone for 4-5 days. In the presence of DHT, JC9 still displayed a good potency to decrease LNCaP cell growth; the magnitude of cell growth decrease in the presence or absence of DHT was actually comparable. HF, at the same test concentration, exerted a moderate activity to repress DHT-stimulated cell growth; this finding is in line with the previously reports that HF is a weak antiandrogen against LNCaP mutant AR. Based upon the above findings, it is concluded that JC9 can effectively nullify prostate cancer cell growth in the presence or absence of male hormones. Since JC9 appears to be more potent than HF, JC9 may have potential to be developed into a drug candidate for prostate cancer disease management.

Western Blot Analysis of AR Expression

AR is the key factor in regulating prostate cancer cells' response to androgens. We examined whether JC9 influences the steady-state level of AR. LNCaP cells were treated with 5 μM of JC9 as mentioned above. At designated times, cell lysate was prepared for Western blot analysis following the previously described conditions. A color detection method was subsequently employed to examine AR and actin protein signals in the membranes, and the resultant protein signals were quantitated by densitometry. In FIG. 2B, normalized AR signals are reported, which were expressed as a percentage of baseline (Day 0) value.

The results show that the endogenous level of AR steadily declined in LNCaP cells treated with JC9. AR reduction was first observed after 2 days of continuous incubation with JC9, and at the end of a 5-day incubation, only ˜10% initial level of AR remained in the treated cells. It is noteworthy that there is a correlation between the decrease in cell number and AR reduction after JC9 incubation. This information strongly suggests that JC9 may act, at least partly, through a AR lowering mechanism to down-regulate LNCaP cell growth.

In Vivo Xenograft Tumor Growth Assay

In related in vivo studies, LNCap human prostate tumor cells are xenografted by subcutaneous injection (2×10⁶ per site) into nude mice. Mice are subsequently treated by intraperitoneal injection of either the vehicle solution alone as a control or the test compounds (JC9)) at a dose of 100 milligrams per kilogram body weight, three times a week for 7 weeks. Tumor volume is measured twice a week over the next 7 weeks. Treatment with compounds such as JC9 or JC15 for a sufficient period of time (for example, from between 2 weeks to a few months) is expected to result in a significantly reduced rate of tumor growth. Such results can be taken as a strong indication that the suppression of AR activity and the resulting reduction of tumor growth induced by AR-degrading compounds such as JC15 or by JC9, may be translated into a practical use for treating or preventing diseases and disorders related to AR activity, such as prostate and other cancers.

### Example 3

### Specificity of Steroid Hormone Receptor Degradation in Different Cell Lines

This describes a non-limiting example of specific degradation of nuclear receptors (in this case, steroid hormone receptors) in various cell lines. Two representative tumor cell lines were used to test the effects of the compound JC15 on the androgen receptor: the human prostate cancer cell line, LNCaP, and the human mammary adenocarcinoma cell line, T47D

Human prostate cancer LNCaP cells and T47D cells were plated at a density of 7×10⁵ cells per 60 millimeter tissue culture dish in Richter's Improved MEM Insulin (RPMI) medium containing 10% FBS. The medium was changed to RPMI or DME medium containing 10% charcoal-stripped serum 24 hours later to deplete cellular androgens or estrogens. After another 24 hours, treatment with the test compounds began. The test dose of JC15 was 1 or 2 micromolar and the test does of JC9 was 1, 5 and 10 micromolar. LNCap cells also received dihydrotestosterone (DHT) (3 nanomolar). Control cells received a corresponding amount of the vehicle, dimethylsulfoxide (DMSO) (<0.04%) for an equivalent exposure time. Cells were incubated with JC15 for 24 or 48 hours, and were lysed in 250 microliters of 1×SDS/PAGE loading buffer. Approximately 40 micrograms of total cellular protein was loaded in each lane of a pre-cast gel (NuPAGE, Invitrogen). Protein separation and transfer were performed following the manufacturer's instructions.

For the prostate cell lysates, androgen receptor (AR) protein was visualized by incubating the resultant membranes with an anti-AR antibody (BD-PharMingen), followed by chemiluminescence detection (ECL Plus, Amersham). To examine the effect of the test compounds on other cellular proteins, several identical gels were prepared and the resulting membranes incubated with antibodies specific for prostate-specific antigen (PSA), glucocorticoid receptor (GR), estrogen receptor beta (ER beta), peroxisome proliferator-activated receptor gamma or beta (PPAR gamma or beta), retinoid X receptor (RXR), the major cellular protein 70-kDa heat shock protein (hsp70), and a cytoskeletal protein, actin. Antibodies for PSA and various nuclear receptors were obtained from Santa Cruz Biochemicals, while the antibodies for hsp70 and actin were from StressGen and Sigma, respectively. The resultant protein signals were quantified using densitometry and NIH ImageJ software.

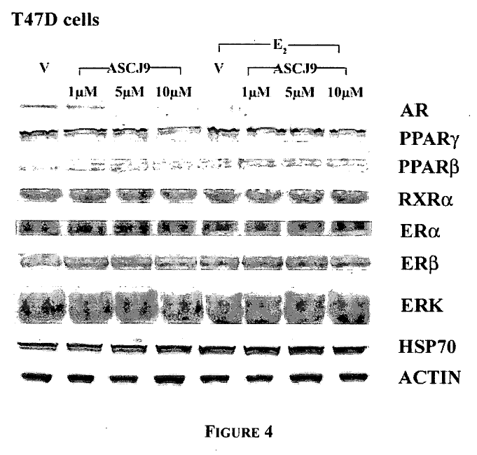

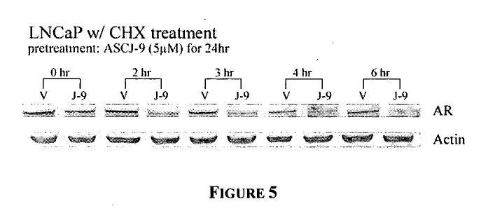

The Western blots of LNCaP cell lysates are shown in FIG. 3. Incubating LNCaP cells with JC15 (1 or 2 micromolar, 24 hours), in the presence or absence of DHT, decreased the cellular concentrations of AR and progesterone receptor, but did not substantially affect that of glucocorticoid receptor, ER beta, PPAR gamma, PPAR beta, retinoid X receptor, hsp70, or actin. PSA levels were also observed to be decreased by JC15 treatment. PSA expression is regulated primarily by the androgen receptor, which induces PSA expression through androgen response element-containing enhancer elements in the PSA promoter (Heinlein & Chang (2004)), and the observed reduction in PSA levels is consistent with the observed decrease in AR levels.

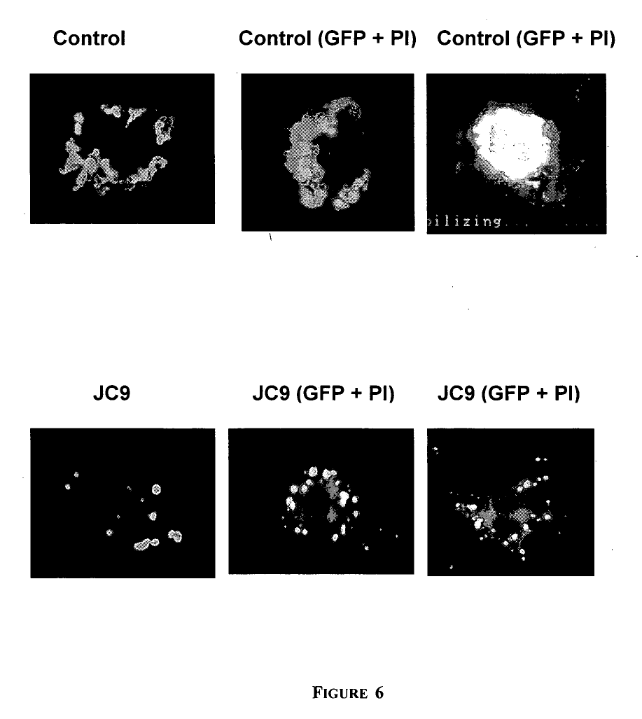

The Western blots of T47D cell lysates are shown in FIG. 4. Incubating T47D cells with JC9 (5 or 10 micromolar) in the presence or absenec of estrodial (E2) decreased the cellular concentrations of AR but not other receptor proteins.

### Example 4

### Enhancing Degradation of a Transcription Factor in the Presence of a Protein Synthesis Inhibitor

To determine whether the observed reduction of AR protein levels was due to protein degradation rather than inhibition of AR protein synthesis, a second set of three replicate experiments was performed. In these experiments, the protein synthesis inhibitor cycloheximide (CHX) was used to prevent the cells from synthesizing new proteins. In the absence of new AR protein synthesis, any alterations in AR levels would be mainly attributable to protein degradation. LNCaP cells were cultured, and incubated with JC9 (5 micromolar) for about 20 hours. Subsequently, cycloheximide (15 micrograms/milliter) was added to the cultured cells, which were then incubated for 0, 2, 3, 4 or 6 hours before harvesting and analysis of AR levels by Western blot.

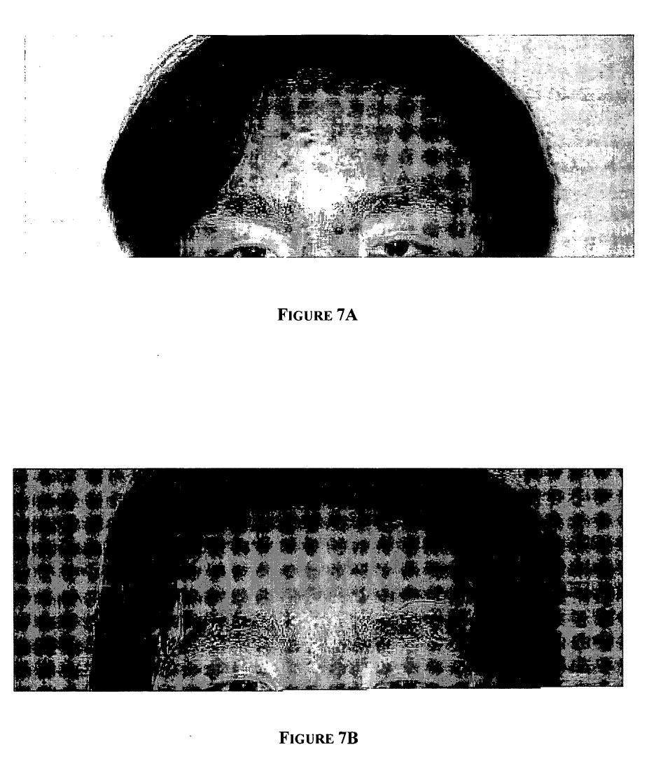

A representative western blot from an experiment is depicted in FIG. 5. A reduction of endogenous AR concentration in the control cells was detected within about 2-3 hours of treatment with cycloheximide, suggesting that de novo AR synthesis contributes to the steady-state level of this receptor. The observed reduction of existing AR protein indicates that the test compound (JC9) enhanced or increased the degradation of existing AR protein (and thus decreased AR activity) within 4 hours or less.

### Example 5

### Degradation of a Mutant Androgen Receptor

This describes a non-limiting example of degradation of a mutant nuclear receptor in a model of a human disease associated with accumulation of the mutant nuclear receptor. In this specific example, a model of Kennedy disease is investigated. As described in detail above under the heading “II. METHOD FOR TREATING STEROID-RECEPTOR ASSOCIATED DISEASES”, Kennedy disease or spinobulbar muscular atrophy (SMBA) is a neurodegenerative disease caused by an androgen receptor mutation consisting of an abnormally long polyglutamine expansion in the N-terminus region of the AR gene. Experimental transfection of cells with a mutated AR having expanded polyglutamine has been shown to be associated with a decreased transactivational function and, in some cases, intranuclear inclusions of misfolded AR proteins (Chamberlain et al. (1994) Nucleic Acid Res., 22:3181-3186). This intranuclear accumulation of abnormal AR is cytotoxic, triggering neuronal cell death, consistent with the in vivo pathology of Kennedy disease.

Monkey kidney COS-1 cells were plated at a density of 3×10⁴ cells per 0.5-milliliter volume onto alcohol-cleaned and sterilized cover slips placed in 35-millimeter suspension culture dishes containing Dulbecco's modified Eagle's (DME) medium containing 10% FBS. The cells were transfected with plasmids containing either the Q19 (plasmid GFPARQ19) or Q49 (plasmid GFPARQ49) mutant androgen receptor and green fluorescent protein (GFP) as a reporter. For each coverslip, 12.3 microliters SuperFect was added to 3.075 micrograms plasmid in 102.5 microliters DME medium (to give a 1:4 ratio of DNA to SuperFect reagent); the mixture was vortexed briefly, and the complex allowed to form over 15 minutes. Each mixture then received 897 microliters CD/DME and was mixed gently. The resulting 1-milliliter volumes were added to the dishes containing the coverslips (final plasmid concentration was 3.02 micrograms per dish). Cells were allowed to incubate with the transfection solution for 5 hours, then the medium was changed to fresh 1.5 milliliters CD/DME medium with either vehicle (DMSO) only added or JC9 (final concentration 5 micromolar). Twenty-four hours after transfection termination, the medium was changed again to fresh CD/DME medium (with or without 1.5 nanomolar DHT), and either vehicle or JC9 added (final concentration 5 micromolar). Twenty-four hours after changing medium, the medium was removed and the cells fixed with 1% formaldehyde in phosphate-buffered saline (PBS) for 1 hour at room temperature. The formaldehyde was removed and the fixed cells washed with PBS three times, and the coverslips then allowed to dry. Coverslips were marked to indicate the treatment scheme and hydrophobic circles made with a wax pencil around the cells. Each coverslip was stained with 200 microliters of propidium iodide (0.7 micrograms per milliliter in water) for 5 minutes at room temperature, then rinsed three times with PBS. The coverslips were air-dried, mounted on slides with a glycerol-based mounting agent, and stored at 4 degrees Celsius if necessary prior to observation with fluorescence microscopy. Representative micrographs showing COS-1 cells transfected with the GFPARQ49 plasmid are depicted in FIG. 6. As shown in the micrographs, transfected cells expressed the plasmid as shown by the fluorescent reporter protein GFP. Control cells contained large amounts of fluorescent inclusions or aggregates. Cells that had been treated with JC9 contained substantially smaller amounts of fluorescent inclusions, suggesting that the expressed mutant Q49 androgen receptor was degraded by JC9 treatment.

### Example 6

### Treatment of an Androgen-Related Disorder in Human Subjects by Degradation of the Androgen Receptor

This example describes the treatment of a nuclear receptor-related disorder (acne vulgaris) in a subject by degradation of the nuclear receptor (the androgen receptor). Acne vulgaris, commonly known simply as acne, is a red skin rash that typically affects the face, chest, and back of teenaged and young adult humans of either sex, though it can occur at any age and on other body areas (see, for example, J. C. Harper and J. Fulton, Jr. (2003), “Acne Vulgaris”, electronically available at www.emedicine.com/derm/topic2.htm, accessed 23 Apr. 2004). Acne affects nearly all people at some point in their life, and can cause permanent scarring and emotional distress and low self-esteem, as well as potentially leading to more severe health problems, such as skin infections. The androgen receptor, which is expressed in the basal cells and glandular cells of sebaceous glands, has a skin distribution that is similar between males and females (Blauer et al. (1991) J. Investig. Dermatol., 97:264-268). In the skin, the androgen receptor stimulates terminal sebocyte differentiation and the production of sebum. Common treatments for acne often have undesirable side effects. For example, topical retinoids can lead to sun sensitivity, antibiotics may result in antibiotic resistance, and benzoyl peroxide can cause contact dermatitis. There is a need for novel and effective, preferably topical (non-systemic), treatments for acne.

In this example, human subjects were successfully treated for acne by topical administration of a cream containing the compound JC15 or JC9, which were shown in the previous examples to ameliorate the effects of the androgen receptor-activated pathway, specifically by inducing degradation of the androgen receptor. A basic carrier formulation was prepared by mixing two solutions: (1) a water-based solution containing aristoflex avc, Osmocide, Tween 20, and water; and (2) an oil-based solution containing isopropyl myristate, coconut dienthanolamine, ethylparaben, isobutylparaben, methylparaben, and propylparaben. The test compounds (JC15 or JC9) were added to the cream to a final concentration of 1 to 2.5 micromolar, as needed.

Male and female human volunteers ranging in age from 15 years to 52 years were treated by topical application of the test compounds to the acne-affected skin. Subjects were asked to apply the cream to the acne-affected areas twice a day (once in the morning and once in the evening). Generally, acne symptoms were observed to significantly subside within 2 to 3 days and completely healed within 1 to 2 weeks. The results are given in Table 1, and a representative result (photo pictures) from one volunteer is depicted in FIG. 7.

TABLE 1

| | | Treatment | | | | |
|---|---|---|---|---|---|---|
| | | | Concen- | | | |
| | | | tration | | Relative | |
| Subject | | Com- | (micro- | | effective- | Recovery |
| Age | Sex | pound | molar) | Frequency | ness | Time |
| 17 | F | ASCJ15 | 1 | Twice a day | ++ | 1 week |
| 15 | M | ASCJ15 | 1 | As often as | +++ | 1 week |
| | | | | needed | | |
| 15 | M | ASCJ15 | 1 | As often as | +++ | 1 week |
| | | | | needed | | |
| 38 | F | ASCJ15 | 1 | As often as | ++ | 2 weeks |
| | | | | needed | | |
| 52 | F | ASCJ15 | 1 | Twice a day | ++ | 2 weeks |
| 42 | F | ASCJ15 | 1 | Twice a day | ++ | 2 weeks |
| 19 | M | ASCJ9 | 1 | Twice a day | + | 2 weeks |
| 18 | F | ASCJ9 | 1 | Four times | +++ | 1 week |
| | | | | a day | | |
| 24 | F | ASCJ9 | 2.5 | Twice a day | +++ | 1 week |

### Example 7

### Reduction of Sebaceous Glands in Rats by Degradation of the Androgen Receptor

In this example, the test compounds JC15 and JC9, which were shown in the previous examples to ameliorate the effects of the androgen receptor-activated pathway, specifically by inducing degradation of the androgen receptor, and to be effective in treating acne in human subjects, were used to reduce sebaceous glandular lobe size in an animal model. Effective reduction of sebaceous glands by topical treatment may be useful in treating skin conditions such as acne. Fuzzy rats were used in this animal model as described in Ye et al. (1997) Skin Pharmacol., 10:10288-10297, which is incorporated by reference in its entirety herein.

Topical creams were prepared as described in EXAMPLE 6. The test creams contained JC9 (25 micromolar) or JC15 (1 micromolar); a control cream with only vehicle added was also prepared. The test or control creams were applied using a cotton swab to the dorsal skin of the animal, once daily, over a period of 8 weeks. Animals were then sacrificed and skin samples collected for microscopic examination. Commercial hair remover was applied to the dorsal surface of the euthanized animals. After 5 minutes, the hair remover and hairs were removed with a tissue. The area was thoroughly cleaned with 75% isopropyl alcohol. A 4-millimeter skin punch was used to remove skin tissue samples, which were incubated for 2 to 3 hours in a ethylenediaminetetraacetate (EDTA, 17 millimolar), sodium phosphate (0.1 molar, pH 7.4) solution at 37 degrees Celsius. The epidermis was carefully separated from the dermis and stored in 10% phosphate-buffered formalin. Prior to microscopic examination, the samples were mounted on glass slides. Areas of well-preserved glandular lobules were selected for microscopic imaging. Edges of the glandular lobes were traced and the areas of the traced lobes obtained with Image J software (National Institutes of Health).

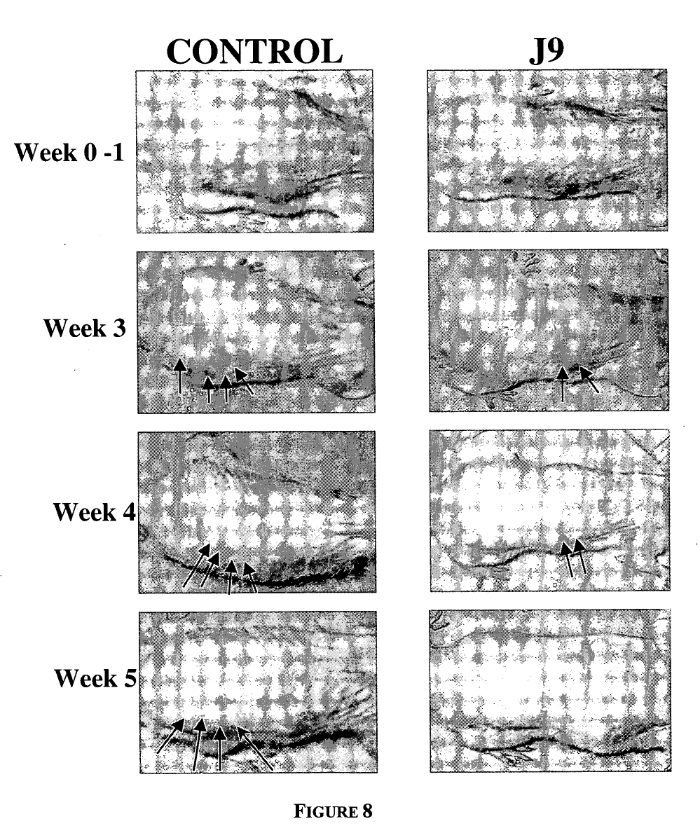

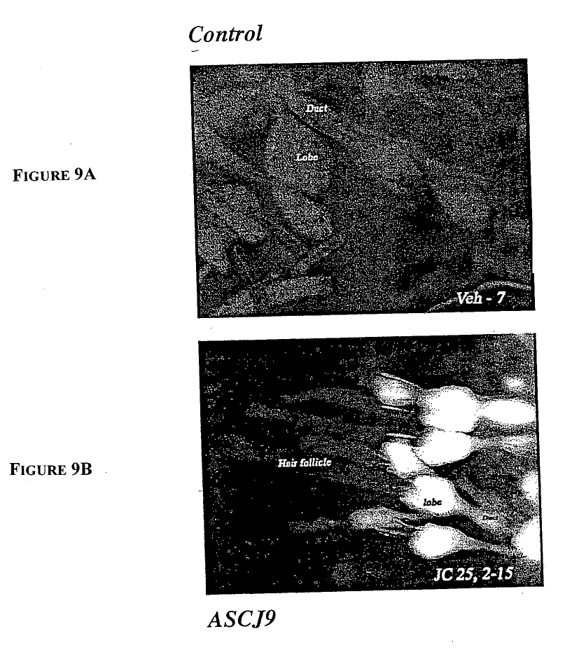

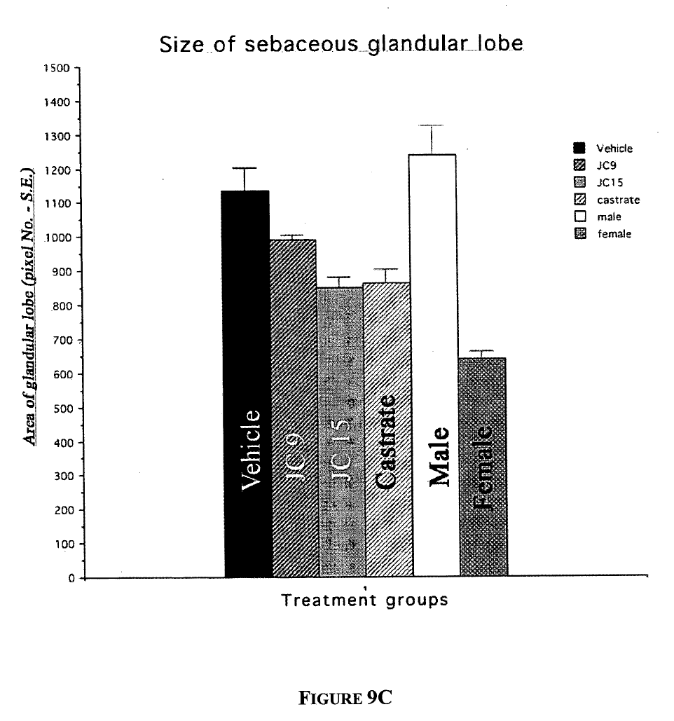

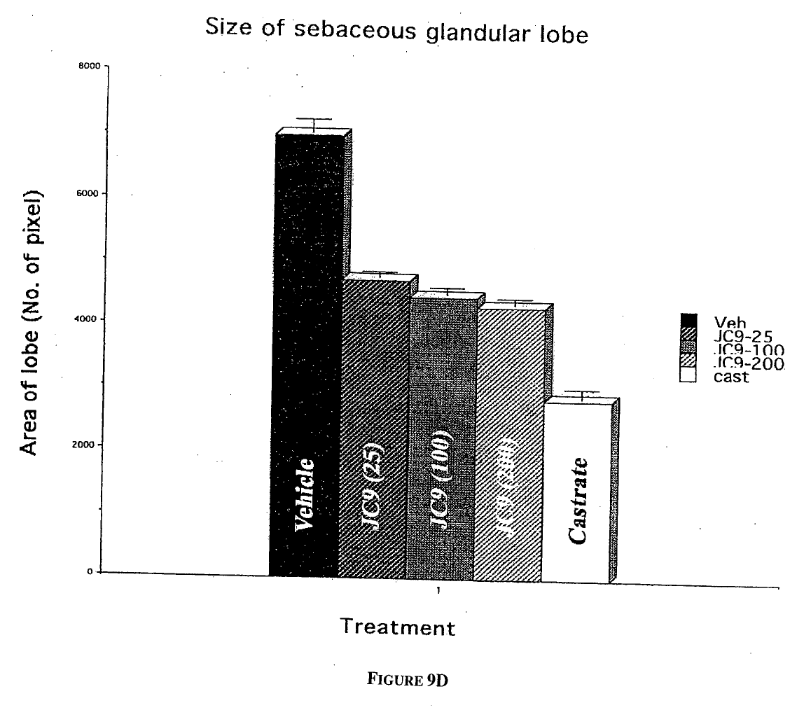

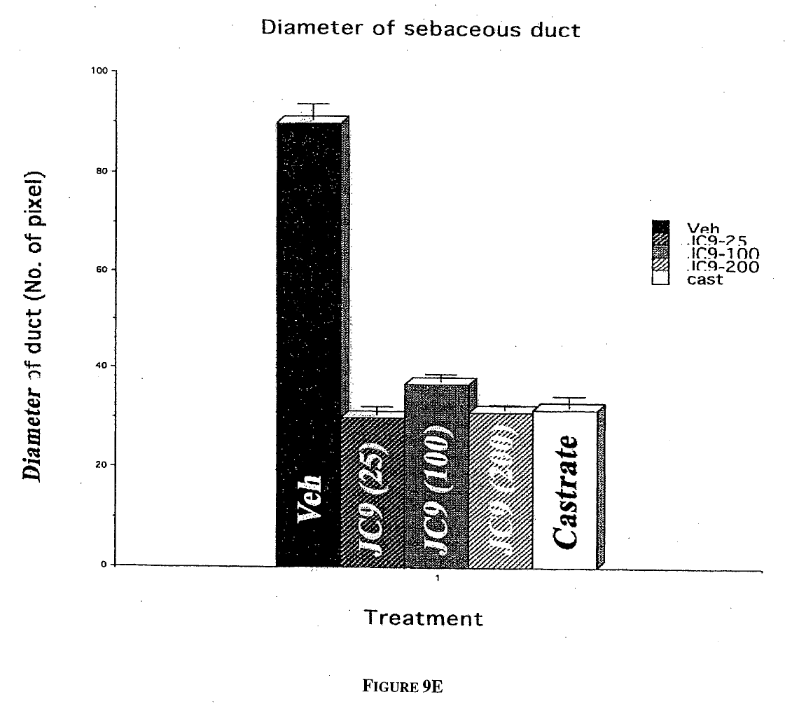

Results are shown in FIG. 8 and FIG. 9. As shown in FIG. 8, the bands of sebaceous glands were reduced within 4-5 weeks in the Fuzzy rats treated with JC9. Similar results (not shown) were observed in animals treated with JC15. As shown in FIG. 9A-E, the size of the sebaceous glandular lobe is approximately twice in male Fuzzy rats as in females. Topical treatment with the vehicle only (control cream) did not produce a significant change in male rats. Topical treatment of male rats with the test compounds JC15 and JC9 resulted in a reduction in the size of the sebaceous glandular lobe and duct, with the decrease caused by JC15 approximately equivalent to the decrease caused by castration.

### Example 8

### Treatment of Androgen-Induced Alopecia in an Animal Model by Degradation of the Androgen Receptor

This example describes the treatment of a nuclear receptor-related disorder in a subject by degradation of the nuclear receptor. In this example, the nuclear receptor is the steroid hormone receptor, the androgen receptor. The nuclear receptor-related disorder is alopecia (hair loss or baldness), which is known to be affected by the androgen receptor. In this example, the test compound JC9, which was shown in the previous examples to ameliorate the effects of the androgen receptor-activated pathway, specifically by inducing degradation of the androgen receptor, is used to treat hair loss in an animal model.

C57BL/6J mice were used in this animal model for hair loss and regrowth (Uno et al. (1990) J. Cutaneous Aging & Cosm. Derm., 1: 193, which is incorporated by reference in its entirety herein). Six-week-old male mice (6 to 7 animals per group) were shaved with an electric clipper, and then treated with a hair-removal cream for 1 to 2 minutes. Animals that were found to have a dark skin color after shaving, indicating that they were in anagen phase where there is active growth of hair follicles, were excluded from the study. One day after hair removal, a first group of animals each received 100 microliters of a 1% testosterone solution in ethanol, applied topically to the shaved area, once each morning for twenty consecutive days. A second group of animals each received 100 microliters of vehicle (ethanol) alone, applied topically to the shaved area, once each morning for twenty consecutive days. The first group of mice (testosterone-treated) were divided further into a control group and a treatment group. Also beginning one day after hair removal, each mouse in the control group received 100 microliters of a control solution (60% ethanol, 20% propylene glycol, and 20% water) and each mouse in the treatment group received 100 microliters of the test compound, JC9 (0.02% in the same 60% ethanol, 20% propylene glycol, and 20% water solution), applied topically to the shaved area, once each afternoon for twenty consecutive days. Hair re-growth in the shaved areas was observed and photographed at 12, 15, 18, 21, and 24 days after beginning of the topical treatments.

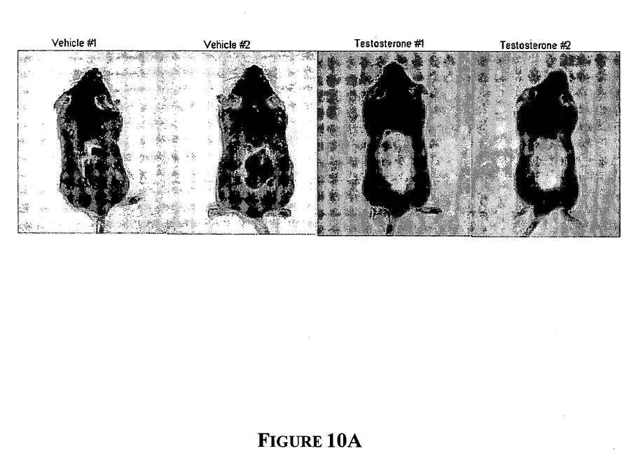

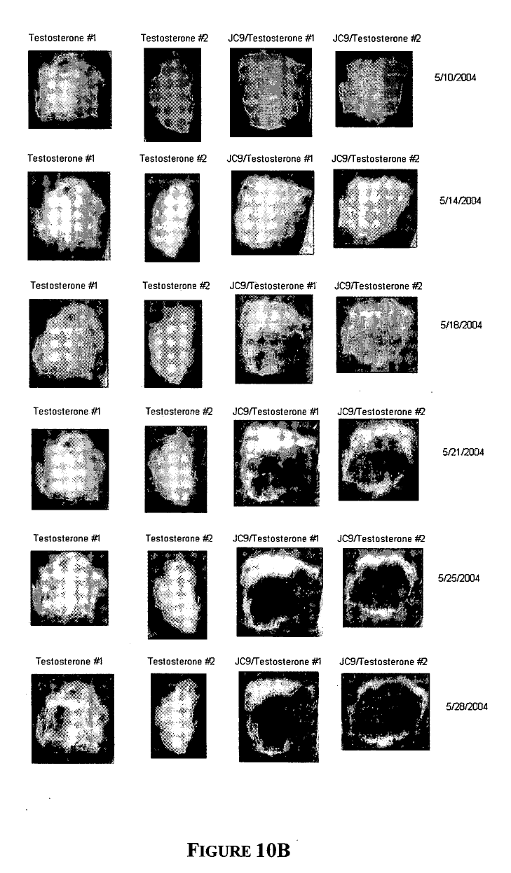

Mice that were shaved and then received topical morning applications of testosterone and afternoon applications of the control solution only showed little or no re-growth of hair in the shaved areas after 20 days of treatment (FIG. 10A). Mice that were shaved and then received topical application of the ethanol vehicle alone (without testosterone) showed rapid re-growth of hair in the shaved areas after 20 days of topical vehicle treatment (FIG. 10A).

Mice that were shaved and then received topical morning applications of testosterone and afternoon applications of the control solution only showed little or no re-growth of hair in the shaved areas after 20 days of treatment (FIG. 10B). Mice that were shaved and then received topical morning applications of testosterone and afternoon applications of JC9 showed rapid re-growth of hair in the shaved areas from day 10 to day 20 (FIG. 10B). These results demonstrate that topical application of JC9, a compound known to degrade the androgen receptor, is able to overcome testosterone-induced hair growth suppression in an animal model.

### Example 9

### In Vivo Reduction of Cancerous Tumor Using Nuclear Receptor Degradation Compound JC9

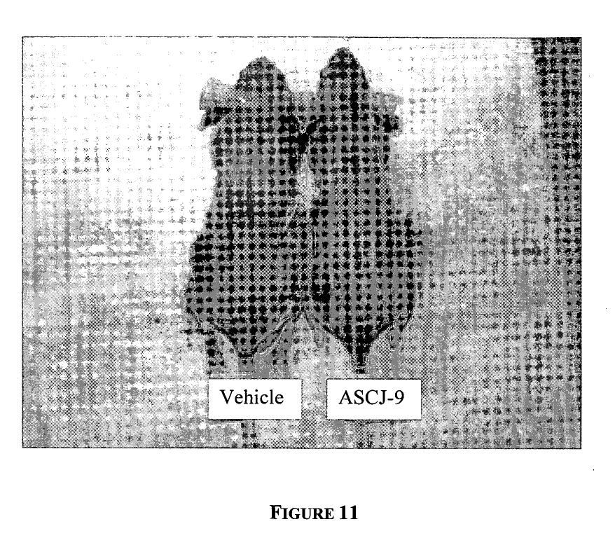

Two million LNCaP tumor cells were inoculated, subcutaneously, into the left flank of nude mice. In the experimental animals, the nude mice were give an intraperitoneal (ip) injection of compound JC9 at 100 mg/kg/day three times per week or with vehicle control only. After 7 weeks of treatment the tumors were excised, weighed and compared. The tumor weight in ratio of vehicle control to JC9 was 0.694 g:0.172 g. Therefore the JC9 treated animal demonstrated a 75% reduction in tumor size. Results are shown in FIG. 11.

All headings are for the convenience of the reader and should not be used to limit the meaning of the text that follows the heading, unless so specified. Various changes and departures may be made to the present invention without departing from the spirit and scope thereof. Accordingly, it is not intended that the invention be limited to that specifically described in the specification or as illustrated in the drawings, but only as set forth in the claims.

What is claimed is:

1. A method of inhibiting a nuclear receptor activation pathway, comprising:
a) providing a cell comprising a nuclear receptor activation pathway; and
b) introducing a compound capable of enhancing degradation of a nuclear receptor in said nuclear receptor activation pathway.

2. The method according to claim 1, wherein said cell is a mammalian cell.

3. The method according to claim 1, wherein said cell is a human cell or a cell derived from a human cell.

4. The method according to claim 1, wherein said nuclear receptor activation pathway comprises a STAT signalling pathway.

5. The method according to claim 1, wherein said nuclear receptor activation pathway is an androgen receptor (AR) pathway or a progesterone receptor (PR) pathway.

6. The method according to claim 1, wherein said nuclear receptor activation pathway is selected from the group consisting of an estrogen receptor (ER) pathway, a glucocorticoid receptor (GR) pathway, a 9-cis retenoic acid (RXR) pathway and a trans-retenoic acid (RAR) pathway.

7. The method according to claim 1, wherein said compound is a curcumin analogue or a curcumin derivative.

8. The method according to claim 7, wherein said curcumin analogue or said curcumin derivative is JC9 or JC15.

9. The method according to claim 7, wherein said curcumin analogue or said curcumin derivative is an analogue or derivative of JC9 or JC15.

10. The method according to claim 1, wherein said compound enhances degradation of said nuclear receptor by a method selected from the group consisting of interfering with phosphorylation of said nuclear receptor, interfering with dimerization of said nuclear receptor, interfering with binding of said nuclear receptor to a cofactor, and interfering with nuclear translocation of said nuclear receptor.

11. A method of inhibiting a STAT activation pathway comprising:
a) providing a cell comprising a STAT activation pathway; and
b) introducing a compound capable of enhancing degradation of a STAT transcription factor protein in said nuclear receptor activation pathway.

12. The method according to claim 11, wherein said STAT transcription factor protein is selected from the group consisting of: STAT1, STAT2, STAT3, STAT4, STAT5a, STAT5b and STAT6.

13. The method according to claim 11, wherein said compound inhibits or reduces an immune response.

14. A cosmetic composition comprising:
a) a compound capable of inducing degradation of a nuclear receptor; and
a) a cosmetically acceptable carrier.

15. The cosmetic composition according to claim 15, wherein said compound is a curcumin analogue or curcumin derivative.

16. The cosmetic according to claim 15, wherein said compound is JC9 or JC15.

17. The cosmetic according to claim 14, wherein said cosmetic is for the prevention or treatment of a skin disorder.

18. The cosmetic according to claim 14, wherein said cosmetic is for the prevention or treatment of a medical condition selected from the group consisting of inflammation, acne, baldness, hirsutism, an exposed wound, a burn, atopic dermatitis, enzema, psoriasis.

19. A cosmetic composition comprising:
a) a compound capable of inducing degradation of a STAT transcription factor protein; and
b) a cosmetically acceptable carrier.

20. The cosmetic composition according to claim 19, wherein said cosmetic is treats or prevents a skin disorder selected from the group consisting of inflammation, acne, atopic dermatitis, enzema and psoriasis.

21. A pharmaceutical composition comprising:
a) a compound or a pharmaceutically acceptable salt thereof capable of enhancing degradation of a nuclear receptor; and
b) a pharmaceutically acceptable carrier.

22. The pharmaceutical composition according to claim 21, wherein said compound is a curcumin derivative or analogue.

23. The pharmaceutical composition according to claim 21, wherein said nuclear receptor is a steroid hormone receptor.

24. The pharmaceutical composition according to claim 23, wherein said steroid hormone receptor is selected from the group consisting of the androgen receptor (AR), the progesterone receptor (PR), the estrogen receptor α (ER α) and the estrogen receptor β (ER β).

25. The pharmaceutical composition according to claim 21, wherein said pharmaceutical is for the prevention or treatment of a medical condition selected from the group consisting of male infertility, Kennedy disease, prostate cancer, breast cancer, liver cancer, bladder cancer, benign prostate hyperplasia, acne, baldness, hirsutism, an exposed wound, and a diabetic ulcer.

26. The pharmaceutical composition according to claim 21, wherein said pharmaceutical composition is useful in preventing conception in females of child-bearing age.

27. The pharmaceutical composition according to claim 21, wherein said pharmaceutical is used as a veterinary contraceptive or for a veterinary still birth procedure.

28. The pharmaceutical composition according to claim 21, wherein said pharmaceutical composition inhibits or reduces an immune response.

29. A method to prevent or treat a cause or symptom of a medical condition that is, at least in part, affected by the activity of a nuclear receptor, comprising administering the pharmaceutical composition of claim 21 to an individual suffering from a cause or symptom or medical condition that is at least in part, affected by the activity of a nuclear receptor.

30. The method of claim 29, wherein said nuclear receptor is a steroid hormone receptor and said medical condition is selected from the group consisting of male infertility, Kennedy disease, prostate cancer, breast cancer, liver cancer, bladder cancer, benign prostate hyperplasia, acne, baldness, hirsutism, slow wound healing, and unwanted pregnancy.
